(19) United States
(12) Patent Application Publication
(10) Pub. No.: US 2008/0220091 A1
(43) Pub. Date: Sep. 11, 2008

(54) REDUCTION OF REACTIVE OXYGEN SPECIES IN CHRONIC WOUND MANAGEMENT

(76) Inventors: Stephen H. Monroe, Portland, OR (US); Matthias Johannes Hoekstra, Amsterdam (NL); Albertus Justus Johannes Van Den Berg, Utrecht (NL)

Correspondence Address: O''Melveny & Myers LLP;IP&T Calendar Department LA-1118, 400 South Hope Street, Los Angeles, CA, 90071-2899, US

(21) Appl. No.: 11/970,158

(22) Filed: Jan. 7, 2008

Related U.S. Application Data
(63) Continuation of application No. 11/649,498, filed on Jan. 3, 2007, now abandoned.
Application No. 11/649,498 is a continuation of application No. 10/744,699, filed on Dec. 23, 2003, now abandoned.
Application No. 10/744,699 is a continuation-in-part of application No. 10/645,410, filed on Aug. 21, 2003, now abandoned.
Application No. 10/645,410 is a continuation-in-part of application No. 10/305,713, filed on Nov. 27, 2002, now abandoned.
(60) Provisional application No. 60/436,197, filed on Dec. 23, 2002.
(60) Provisional application No. 60/334,337, filed on Nov. 29, 2001.

(51) Int. Cl. A61K 33/00; A61K 33/30; A61P 17/02; A61K 33/06

(52) U.S. Cl. 424/642; 424/682; 424/722

(57) ABSTRACT

Reactive oxygen species associated with a wound are modulated through treatment of the wound with a solution of metal ions selected from the group consisting of potassium ions, zinc ions, calcium ions and rubidium ions, at a pH of between about 5 and about 7. Preferably, citric acid is employed to adjust the pH of the solution. Application of the extract to a wound exhibiting superoxide anions has been found to be effective in the treatment of these wounds through the reduction of the level of superoxide anions. Moreover, treatment of partial thickness excision wounds as well as contact burn wounds with the present composition has been found to improve epithelialization of these wounds. In addition to the antioxidant activity of the present invention, treatment of the wound employing the present composition produces inhibitory effects on ROS production by human PMNs and on human complement activation, and therefore, is further beneficial in chronic wound management.

### RELATED APPLICATIONS

This application is a continuation of application Ser. No. 11/649,498, filed Jan. 3, 2007, which is a continuation of Ser. No. 10/744,699, filed Dec. 23, 2003, which application is a non-provisional application claiming priority based on Provisional Application Ser. No. 60/436,197, filed Dec. 23, 2002; and is a continuation-in-part application of copending application Ser. No. 10/645,410, filed Aug. 21, 2003, which is a continuation-in-part of copending application Ser. No. 10/305,713, filed Nov. 27, 2002, which is a non-provisional application claiming priority based on provisional application Ser. No. 60/334,337, filed Nov. 29, 2001.

### STATEMENT REGARDING FEDERALLY SPONSORED RESEARCH OR DEVELOPMENT

Not Applicable

### FIELD OF INVENTION

This invention relates to management of wounds, particularly chronic (non-responding) wounds in the nature of decubitus ulcers, burns, and the like.

### BACKGROUND OF INVENTION

In recent years it has also become evident that free radicals play an important role in impaired wound healing. In local and chronic wounds, free radicals are known to cause cell damage and may function as inhibitory factors in the healing process. In chronic wounds, ischemic conditions may convert the enzyme xanthine dehydrogenase into xanthine oxidase which catalyses the conversion of oxygen into superoxide anion. Superoxide anions are also produced in the wound bed by stimulated polymorphonuclear neutrophils (PMNs). Superoxide anion is a free radical toxic to tissue and its generation also results in the formation of other reactive oxygen species (ROS) including the even more toxic hydroxyl radical and the strong non-radical oxidant hypochlorous acid. With nitric oxide, a radical produced by macrophages—another inflammatory cell in the wound bed—superoxide anion easily reacts to form peroxynitrite that also exerts most detrimental effects on surrounding tissue. Finally, superoxide anion may also induce cross-linking of the matrix molecules fibrin and fibronectin resulting in a transformed matrix less suitable for epithelial outgrowth.

### BRIEF DESCRIPTION OF FIGURES

FIG. 1 is a graphic comparison of the IC50 values of a natural oak bark extract and the synthetic solution of the present invention as determined by superoxide anion scavenger assay;

FIG. 2 is a graphic comparison of the IC50 values of a natural oak bark extract and the synthetic solution of the present invention as determined by chemiluminescence assay; and,

FIG. 3 is a graphic comparison of the IC50 values of a natural oak bark extract and the synthetic solution of the present invention as determined by complement assay classical pathway.

### SUMMARY OF INVENTION

In accordance with one aspect of the present invention, reactive oxygen species associated with a wound are modulated upon treatment with a synthetic composition of metal ions. Application of the composition to a wound exhibiting superoxide anions has been found to be effective in the treatment and healing of these wounds through the reduction of the level of superoxide anions associated with the wound. The present invention is particularly effective in the treatment and healing of chronic wounds. Moreover, treatment of partial thickness excision wounds as well as contact burn wounds with the present composition has been found to improve epithelialization of these wounds.

In addition to the antioxidant activity of the present invention, treatment of the wound employing the present composition produces inhibitory effects on ROS production by human PMNs and on human complement activation, and therefore, is further beneficial in chronic wound management.

### DETAILED DESCRIPTION OF INVENTION

Technology and Methods Employed in the Invention

Material

A preferred composition useful in the present invention comprises 10-80 parts by weight of potassium ions, 0.00001-20 parts by weight of zinc ions, 0.01-10 parts by weight of calcium ions, and, rubidium ions I an amount of up to 40 parts by weight, the solution having a pH of between about 5 and about 7. In one embodiment, the metal ions are derived from respective salts thereof, including chlorides, sulfates, citrates, hydroxides, for example. Adjustment of the pH of the composition preferably is accomplished by the addition of citric acid to the solution, as needed. For present purposes, this composition is at times herein referred to as PHI5 (polyhydrated ionogen adjusted to a pH of 5 using citric acid).

Therapeutic value has been found using potassium, zinc and rubidium ions, without calcium. Calcium, however, may be useful in the treatment of certain types of wounds and its presence in a solution of the present invention, even if non-pharmaceutically effective for a particular wound, is not detrimental to the effectiveness of the preferred solution when treating such particular wound.

Assay for Inhibition of ROS Production by Human Neutrophils (Chemiluminescence Assay)

Polymorphonuclear neutrophils (PMNs) were isolated from venous blood of healthy volunteers (Bloedbank Midden-Nederland, Utrecht, The Netherlands). In white 96-well, flat-bottom microtiter plates (Costar, Badhoevedorp, The Netherlands), test samples were serially diluted to final volumes of 50 μL. To each well, 50 μL of PMN suspension (1-10⁷ cells/mL) and 50 μL of luminol (120 μM) were added. PMNs were triggered by adding 50 μL of opsonized zymosan A (OPZ; final concentration: 200 μg/mL. Chemiluminescence was monitored every 2 min for 0.5 sec during a 30-min period using a Titertek Luminoskan luminometer (TechGen International, Zellik, Belgium).

Peak levels were used to calculate the activity of test samples in relation to their corresponding controls (identical incubations without test sample). Experiments were performed in Hank's balanced salt solution (I-IBSS) buffered at pH 7.35 with NaHCO₃ and supplemented with 0.1% (w/v) gelatin to avoid cell aggregation (HBSS-gel). OPZ was obtained by incubation of washed commercial zymosan A with 1:10 diluted human pooled serum (HPS) at 37° C. for 30 min. After washing, the opsonized product was resuspended in HBSS (final concentration: 0.8 mg/mL).

### Superoxide Anion Scavenging Assay

In white, 96-well flat-bottom microtiter plates, test samples were serially diluted in phosphate—buffered saline (PBS; pH 7.4) to a final volume of 50 μL. Subsequently, hypoxanthine (50 μL; final concentration 1 mM), and either buffer or superoxide dismutase (SOD; 25 μL; 10 U/mL) were added. Superoxide anion .O₂ (radical production was initiated by addition of 25 μL of xanthine oxidase (10 mU/mL) and chemiluminescence was monitored every mm for 0.5 sec during 15 min, using a Fluoroskan Ascent FL luminometer (Labsystems, Breda, The Netherlands). Activity of the test compounds was calculated from the SOD-inhabitable part of the chemiluminescence signal. To exclude direct effects of test samples on xanthine oxidase activity, uric acid formation was determined spectrophotometrically at 290 nm.

### Hemotytic Assays for Human Complement Activity (Classical Pathway and Alternative Pathway)

Inhibitory activities of test samples towards the classical and alternative pathways of human complement (CP and AP, respectively) were determined by a modified version of the micro assay described by Klerx et al. (Klerx, J. P. A. M., Beukelman, C. J., Van Dijk, H. et al., Microassay for colorimetric estimation of complement activity in guinea pig, human and mouse serum. J. Immunlol Methods 1983, 63: 215-220). In U-well microtiter plates (Greiner Labortechnik, Nortingen, Germany), test samples were serially diluted in (1) VSB-CP (Veronal saline buffer, prepared with 5 mM veronal, 150 mM saline; pH 7.35), supplemented with 0.15 mM Ca²⁺ and 0.5 mM Mg²⁺ to final volumes of 50 μl (CP) or (2) VSB-AP, prepared with veronal saline buffer as above, supplemented, however, with 0.5 mM Mg²⁺ and 0.8 mM EGTA, to final volumes of 100 μl (AP). Subsequently, 50 μl (CP) or 25 μl (AP) of appropriate dilutions of human pooled serum (HPS; obtained from healthy donors) were added and the plates were incubated at 37° C. for 30° C. min. After addition of 50 μl of sensitized sheep erythrocytes (CP) or 25 μl of rabbit erythrocytes (see below), the plates were incubated again at 37° C. for 1 h. Sheep or rabbit blood in Alsever solution served a sources of erythrocytes. Before use, erythrocytes were washed three times with saline. Sheep erythrocytes were sensitized by incubation with diluted amboceptor (1:800) for 10 min; after washing the sensitized erythrocytes were resuspended in VSB-CP (4×10⁸ cells/ml). Rabbit erythrocytes were suspended in VSB-AP (3×10⁸ cells/ml). Finally, the microtiter plates were centrifuged (900×g, 5 min to spin down intact cells and debris, and 50 μl of the supernatants were transferred to 96-well flat-bottom microtiter plates containing 200 μl of water per well. In the latter plates, the amount of hemoglobin released by lysis of erythrocytes was measured spectrophotometrically using the automatic ELISA reader described above, operated at 405 nm. Controls consisted of similarly treated supernatants of erythrocytes incubated with water (100% hemolysis), or buffer (VSB-CP or VSB-AP; 0% hemolysis), and incubates in which HPS was replaced by heat-inactivated HPS (56° C., 30 min; correction for the background color of test samples).

### Determining Cytotoxicity

A stock solution of 5-carboxyfluorescein diacetate (CFDA; 10 mg/ml) in acetone was prepared and stored at −20° C. Prior to use, this stock solution was diluted 1:1000 in buffer. Propidium iodide (PI; 1.5 mg) was dissolved in 10 ml of phosphate-buffered saline (PBS) containing 2.5% quenching ink, 5% w/v EDTA, and 8 mg of bovine serum albumin (BSA). PMNs were labeled with the vital stain CFDA (10 μg/ml) at 20° C. for 15 min, washed, and resuspended in buffer to a concentration of 10⁷ cells/mL. Amounts of 100 μl of this cell suspension were incubated with equal volumes of diluted samples at 37° C. for 15 min. Subsequently, the cells were washed and stained with 25 μl of PI/ink solution for discrimination between viable (green-fluorescent) and dead (red-fluorescent) cells. The percentage of dead cells was determined using a fluorescence microscope (Fluovert, Leitz, Wetzlar, Germany).

### Results and Discussion

Production of reactive oxygen species associated with a wound may originate from several potential sources. Solutions of the present invention exhibited pharmaceutically effective inhibitory effects on the production of reactive oxygen species (ROS) associated with such wounds.

One source of ROS in a wound are reactive oxygen species generated by stimulated human polymorphonuclear neutrophils (PMNs). PMNs recruited for instance to the wound site and activated, consume increased amounts of oxygen that is converted into ROS. This process known as the respiratory burst is dependent on the enzyme NADPH oxidase that can be activated by both receptor-mediated and receptor-independent processes. Typical receptor-dependent stimuli are e.g. complement components C5a, and C3b, the bacterium-derived chemotactic tripeptide fMLP, and opsonized zymosan; receptor-independent stimuli include long-chain unsaturated fatty acids. Upon activation of the PMN, the multi-component NADPH oxidase is assembled in the cell membrane. Subsequently, the oxidase transfers electrons from NADPH at the cytosolic side of the membrane to molecular oxygen at the other side of the membrane. This results in the generation of superoxide anions (.O₂— either in (intracellular) phagosomes containing ingested microorganisms, or extracellularly. Most of the superoxide anions formed are converted into hydrogen peroxide (H₂0₂). The latter is bactericidal only at high concentrations, whereas superoxide anions themselves do not kill bacteria because of their limited membrane permeability. Some hydrogen peroxide is converted into extremely reactive hydroxyl radicals by the iron-catalyzed Fenton reaction. However, most of the hydrogen peroxide is converted into hypochlorous acid (HOCl), the most bactericidal oxidant known to be produced by the PMN. The latter conversion occurs in the presence of halide (chloride) ions and is catalyzed by myeloperoxidase (MPO), an enzyme also released by activated PMNs. Although in the phagolysosome, intracellular ROS—together with proteolytic and other cytotoxic enzymes released from lysosomes (granules)—serve to kill ingested bacteria and prevent wound infection, extracellular generation of these oxygen metabolites have detrimental effects on surrounding tissue.

In addition to the ROS mentioned above, also the production of nitric oxide (N0. by macrophages present at the wound site) is noted. The radical nitric oxide may easily react with superoxide anion, which results in the formation of peroxynitrite (ONOO), a very potent, relatively stable oxidant with properties similar to those of the hydroxyl radical (see above).

Concerning the inhibition of ROS production by stimulated human PMNs, an IC50 value of 12±2 ml/ml was determined for PHI5, employing Chemiluminescence assay. {(The IC50 value is the sample concentration in the test system giving 50% inhibition; IC50 values represent the mean ± SD (standard deviation) of determinations obtained with two batches of PMNs from two different donors)). Since inhibitory effects in the assay for ROS production may be caused by cell death, also cytotoxic effects of the test samples were investigated. Resting PMNs were labeled with the vital stain CFDA (5-carboxyfluorescein diacetate) and incubated with PHI5. Subsequently, dead cells were stained with propidium iodide. It was found that incubation with 100 μl/ml PHI5 did not show any cytotoxic effects towards PMNs in comparison to control cells, It was concluded that inhibition of ROS production by PHI5, is not due to cytotoxic effects towards PMNs.

Besides generation of superoxide anions by stimulated PMNs as outlined above, these radicals may also arise in chronic wounds where ischemic conditions may convert the enzyme xanthine dehydrogenase into xanthine oxidase which catalyses the conversion of oxygen into superoxide anions. So, antioxidant activity including scavenging of superoxide anions, either produced by the PMN or through xanthine oxidase is regarded beneficial in the treatment chronic wounds. PHI5 was shown to be a significant scavenger of superoxide anions mainly due to the presence of citric acid.

Inhibition as found in the assay for ROS production (see above) may also be caused by a specific scavenging of superoxide anions. Oak bark extract (OBE) has been reported to have a direct effect on PMN functioning. The increase in activity as observed in superoxide anion scavenger assays for employing PHI5 (IC5 O12 μl/ml) is most probably due to additional scavenging of superoxide anions by the citric acid component of PHI5, Thus, PHI5 provides both superoxide anion-scavenging and inhibition of ROS production thereby enhancing the usefulness of the present invention in wound management, particularly management of chronic wounds.

PHI5 also was tested in the hemolytic assays for modulation of complement activity. The complement system is part of the non-adaptive humoral immune system and plays an important role in the human defense mechanism. The complement system comprises over twenty proteins, including complement components C1 to C9. Activation of complement via either the classical, alternative, or lectin pathway results in proteolytic cleavage of the successive complement proteins in a cascade-like manner which eventually leads to the formation of the high-molecular membrane attack complex (MAC) that causes death of bacteria (or foreign red blood cells through lysis). In addition, small split products are generated which mediate many immunoregulatory effects. In this respect, complement factor C3b has a major biological function since (pathogenic) microorganisms and foreign cells (zymosan) are covered with C3b (opsonization), which enables phagocytes with receptors for C3b on their membrane (e.g. PMNs) to recognize, and ingest these invaders and to destroy them by producing ROS. Fragment C5a is another activating agent for PMNs; in addition it is a major chemotactic factor for these phagocytes.

Inhibition of complement activation limits the generation of complement split products such as C5a. As outlined above, this will result in less influx and decreased stimulation of PMNs in the wound bed, and thus reduced extracellular formation of ROS as well as peroxynitrite, and therefore reduced tissue damage.

Although other factors governing wound healing may also be of importance (e.g. MMPs), PHI5 inhibits human complement activation via the classical pathway and inhibits production of ROS by activated PMNs. In addition, citric acid associated with PHI5 has been found to be a contributor to the scavenging of superoxide anions. Such reduction of levels of ROS contribute to the beneficial effects observed in wound management, especially chronic wound management, with preparations containing the metal ions and citric acid of the composition of the present invention.

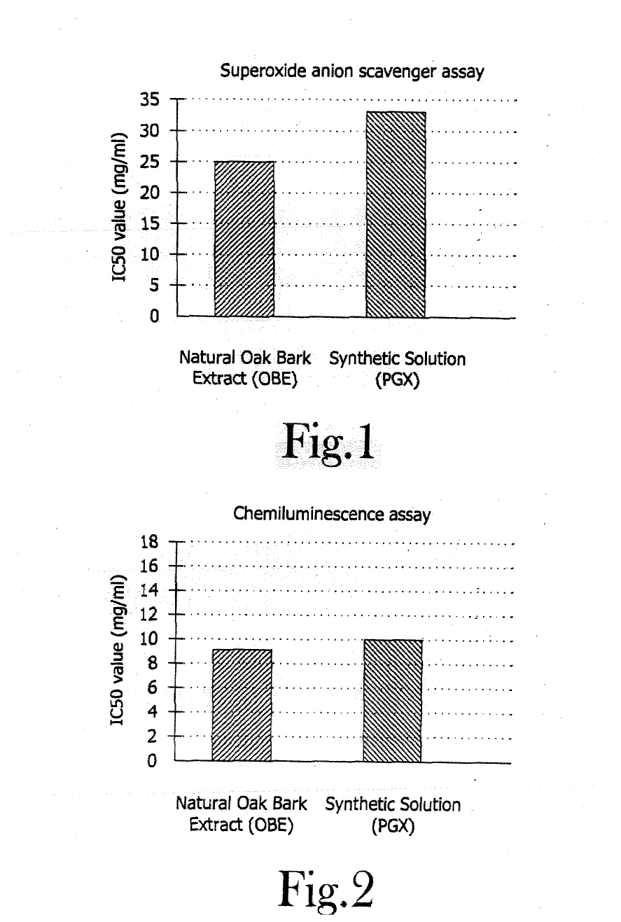

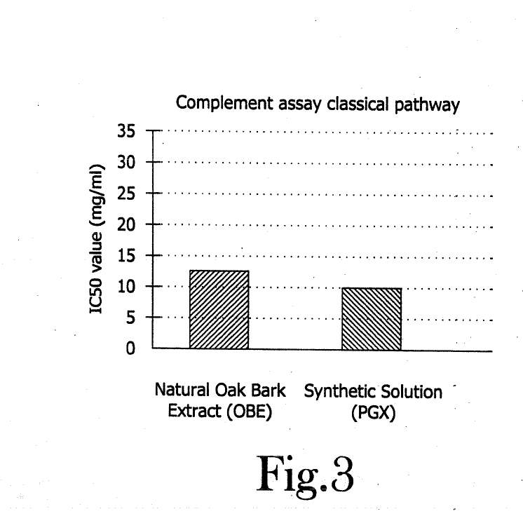

Comparison of the IC-50 values of an oak bark extract of the prior art and the synthetic composition of the present invention are given in FIGS. 1-3 which are graphs depicted to IC50 values of these two compositions as determined in superoxide anion scavenger assay (FIG. 1), chemiluminescence assay (FIG. 2), and complement assay classical pathway (FIG. 3). Review of these graphs shows that PHI5 is more effective than a natural oak bark extract (OBE) with respect to superoxide scavenging and PMN inhibition (chemiluminescence assay), and is only slightly less effective with respect to modulation of complement activity.

What is claimed:

1. A method for the enhancement of the healing of a wound comprising the steps of
providing a pharmaceutically effective aqueous solution of metal ions selected from the group consisting of potassium ions, zinc ions, calcium ions and rubidium ions at a substantially neutral pH, applying said solution to said wound for a time sufficient to effect neutralization of reactive oxygen species associated with said wound, whereby healing of said wound is enhanced by the neutralizing action of said solution upon said reactive oxygen species associated with said wound.

2. The method of claim 1 including the step of adjusting the pH of said solution with citric acid.

3. The method of Claim 2 wherein the pH of said solution is about 5 and about 7.

4. The method of claim 1 wherein said solution exhibits a superoxide scavenging IC50 value of about 33/ml.

5. A method for enhancing the healing of a wound comprising the steps of
providing a pharmaceutically effective aqueous solution of metal ions selected from a group consisting of potassium ions, zinc ions, calcium ions and rubidium ions at a pH of between about 5 and about 7, applying said solution to said wound, whereby said solution effects either inhibition of the production of reactive oxygen species associated with said wound by stimulated polymorphonuclear neutrophils, scavenging of superoxide anions, inhibition of factors that attract or stimulate polymorphonuclear neutophils, or a combination of said actions, and the wound heals.

6. The method of claim 5 wherein the pH of said solution is adjusted to about 5 employing citric acid.

7. The method of claim 5 wherein the pH of said solution is adjusted to about 7 employing hydrochloric acid.

8. The method of claim 1 wherein said solution includes 10-20 parts by weight of potassium ions, 0.00001-20 parts by weight of zinc ions, and rubidium ions in an amount of up to about 40 parts by weight.

9. The method of claim 8 wherein said solution contains sufficient citric acid to adjust the pH of said solution to between about 5 and 7.

10. The method of claim 8 and including 0.01-10 parts by weight of calcium ions.

11. The method of claim 10 wherein said solution contains sufficient citric acid to adjust the pH of said solution to between about 5 and 7.

12. The method of claim 5 wherein said solution includes 10-20 parts by weight of potassium ions, 0.00001-20 parts by weight of zinc ions, and rubidium ions in an amount of up to about 40 parts by weight.

13. The method of claim 12 wherein said solution contains sufficient citric acid to adjust the pH of said solution to between about 5 and about 7.

14. The method of claim 5 and including 0.01-10 parts by weight of calcium ions.

15. The method of claim 5 wherein said solution contains sufficient citric acid to adjust the pH of said solution to between about 5 and about 7.
